(19) United States
(12) Patent Application Publication
(10) Pub. No.: US 2025/0161620 A1
(43) Pub. Date: May 22, 2025

(54) FRAME ASSEMBLY DIRECTLY CONNECTED TO A FRAME AND A SHORT TUBE

(71) Applicant: DCSTAR INC., New York, NY (US)

(72) Inventor: David LUO, New York, NY (US)

(21) Appl. No.: 18/515,499

(22) Filed: Nov. 21, 2023

(51) Int. Cl. A61M 16/06

(52) U.S. Cl. CPC A61M 16/0683

(57) ABSTRACT

A frame assembly directly connected to a frame and a short tube, including a frame and a gas delivery hose and side connectors connected to the frame. The frame is configured to bend towards the face. The frame includes a fixed opening and first connection parts connected to the side connectors. The gas delivery hose includes a first end, a middle hose, and a second end. The side connectors have a front end and a rear end. The front end is for connecting to the frame, the rear end to the face. The frame assembly directly connected to a frame and a short tube also includes an exhaust channel, located on the frame or the gas delivery hose. The frame is detachably connected directly to the gas delivery hose. Combining the exhaust channel with the frame or gas delivery hose reduces the noise and lightens the weight by reducing parts, enhancing the comfort and user experience.

### TECHNICAL FIELD

The disclosure pertains to the field of medical device technology, specifically involving a frame assembly directly connected to a frame and a short tube, suitable for the diagnosis, treatment, and improvement of one or multiple respiratory system diseases.

### BACKGROUND

Obstructive Sleep Apnea OSA is a common sleep-breathing disorder, affecting at or between 4% of men and 2% of women globally. It is characterized by repetitive interruptions in breathing during sleep, with each pause lasting more than 10 seconds, which can occur dozens or even hundreds of times in one night. These pauses are due to the repeated collapse of the patient's upper airway during sleep, and each collapse can lead to a drop in blood oxygen saturation and may trigger a series of physiological and psychological responses, including arousal, rapid heartbeat, cardiovascular diseases, daytime sleepiness, and mental fatigue. If not treated in a timely manner, it can severely impact the patient's health and quality of life.

Although there are many patients with sleep-breathing disorders, the majority cannot maintain long-term treatment due to issues related to the comfort, ease of use, stability, adaptability, sealing, and noise of the therapeutic equipment. The complex three-dimensional shape of the face, combined with the varying bone structures in different individuals, poses a significant challenge in disclosing a mask that seals effectively. One primary challenge is ensuring the mask both effectively seals the patient's airway and fits well with the frame. Most current mask systems use a combination of nasal pillow, frame, elbow, and straps, aiming to secure the nasal pillow to the patient's face through a rigid frame combined with straps. The weight of the frame, coupled with the weight of the matching elbow, can make some mask systems heavy. This can lead to red marks and pressure sores on the face, decreasing comfort. Furthermore, it is more complex for users to use if the mask systems require assembly and/or complex assembly. To alleviate the discomfort brought by the mask, some smaller patient interfaces, for example, nasal cannulas, are sold in the market. These types of interfaces might be more comfortable due to their lightweight design but may lack stability. During sleep, users may move their heads, facial bones, and muscles, so an unsecured tube might fall off or leak, leading to suboptimal treatment outcomes.

Therefore, a new frame assembly directly connected to a frame and a short tube is needed to overcome the limitations of current devices and provide a more stable, simpler, and user-friendly solution. While retaining the primary functionalities of the product, this disclosure describes a device that would enhance the user experience, elevate the overall comfort of the product, make it easier to clean, and significantly extend the product's overall lifespan.

### SUMMARY

The objective of this disclosure is to provide a new type of a frame assembly directly connected to a frame and a short tube to overcome the present limitations of current technology, and provide a solution which is more efficient, portable, and user-friendly, catering to the effective treatment needs of patients with obstructive sleep apnea.

In an embodiment, a frame assembly directly connected to a frame and a short tube is provided. The frame assembly directly connected to a frame and a short tube comprises: a frame for securing a nasal pillow, a gas delivery hose connected to the frame for delivering breathable gas to the nasal pillow, and side connectors for securing the frame to a user's face. The frame is configured to accommodate the nasal pillow and position the nasal pillow at the user's nostrils, in which at least a part of the frame is rigid, and the frame is configured to bend towards the user's face such that the frame fits to the user's face and ensures a secure attachment of the nasal pillow to the user's face.

The frame includes:
a fixed opening located in a middle of the frame, in which the frame connects to the gas delivery hose through the fixed opening, and the fixed opening is a tubular body of a certain length, providing sufficient contact surface for the connection of the gas delivery hose to the fixed opening;
first connection parts located horizontally on both sides of the frame with the frame connecting to the side connectors via the first connection parts.

The gas delivery hose includes:
a first end located at an upper part of the gas delivery hose for detachable connection to the frame, in which the gas delivery hose is configured to rotate relative to the frame once the gas delivery hose is connected to the frame;
a middle hose positioned between the first end and a second end, and connected to both the first end and the second end, the middle hose having multiple continuous evenly folded walls, enabling the middle hose to bend and stretch;
the second end located at a lower part of the gas delivery hose and configured to connect with an other tubular connector; the second end consisting of a first part and a second part, in which the first part is fixedly connected to a lower end of the middle hose, with an other end of the first part away from the middle hose connected to the second part, and an other end of the second part connects with the other tubular connector, thereby being configured to deliver breathable gas to the gas delivery hose, which is then channeled through the frame and the nasal pillow to the user's nostrils.

The side connectors connect to the first connection parts on both sides of the frame; and the side connectors consists of a front end and a rear end, the front end configured to connect with the frame, while the rear end is configured to make contact with the user's face.

The frame assembly directly connected to a frame and a short tube further includes an exhaust channel, which is located on the frame and configured to allow for the expulsion of exhaled gas from the user's nostrils through the nasal pillow. And the gas then flows into the frame and exits via the exhaust channel on the frame, ensuring the user inhaling fresh air promptly.

In an embodiment, the fixed opening has a central axis located in the middle of the fixed opening. The frame is a symmetrical shape with left-right symmetry along a straight line which is the axis of symmetry for the frame. And the central axis and the axis of symmetry have at least one of the following characteristics: A. An angle α, formed by the central axis of the fixed opening and the straight line connecting the furthest point on the frame from the fixed opening and the center point defined by the central axis of the fixed opening, is at or between 0° to 90° towards the user's face; B. The angle β, formed by the axis of symmetry of the frame and the central axis of the fixed opening, is at or between 30° and 150°.

In an embodiment, a length of the gas delivery hose is at or between 200 to 600 mm, an external diameter of the gas delivery hose is at or between 25 to 45 mm, and a wall thickness of the gas delivery hoses is at or between 1 to 5 mm, with an expandability of the gas delivery hose being at or between 80% to 120%.

In an embodiment, the connections between the gas delivery hose and the frame, the side connectors and the frame, and the nasal pillow and the frame includes one or more of buckles, snap fasteners, hook and loop fasteners, rotary knobs, or magnetic attraction fasteners.

In an embodiment, there are multiple exhaust channels on the frame. A combined area of the exhaust channels constitutes 20%-50% of an outer surface area of the frame.

In an embodiment, the exhaust channel is positioned towards a side of the gas delivery hose, includes a noise reduction piece that covers the exhaust channel and is configured to reduce noise when the frame vents to an external environment through the exhaust channel.

In an embodiment, the exhaust channel includes exhaust grids or exhaust holes, and the exhaust grids are made of through holes opened on a surface of the frame and are attached to the frame to cover the through holes by adhesive, buttons, ultrasonic bonding, or heat pressing; and the exhaust holes are formed by multiple small circular holes on the surface of the frame.

In an embodiment, the second part of the second end of the gas delivery hose is a cylinder that is configured to rotate coaxially relative to the first part, allowing the second part to achieve coaxial rotation relative to the gas delivery hose.

In an embodiment, the front end and rear end of the side connectors are integrally formed through injection molding, and a linear distance between a center point of the front end and a center point of the rear end is at or between 20 to 200 mm.

In an embodiment, the noise reduction piece includes one or more of noise-reducing cotton or noise-reducing mesh, and the noise-reducing cotton is made from at least one material selected from polyester, polypropylene, polyethylene, nylon, vinylon, and natural fabric; and the noise-reducing mesh is made from at least one material selected from polyvinyl chloride, polypropylene, polytetrafluoroethylene, or nylon.

In an embodiment, a frame assembly directly connected to a frame and a short tube is provided. The frame assembly directly connected to a frame and a short tube includes a frame for securing a nasal pillow, a gas delivery hose connected to the frame for delivering breathable gas to the nasal pillow, and side connectors for securing the frame to a user's face. The frame is configured to accommodate the nasal pillow and position the nasal pillow at the user's nostrils with at least a part of the frame being rigid, and the frame is configured to bend towards the user's face such that the frame fits to the user's face and ensures a secure attachment of the nasal pillow to the user's face;

The frame includes:
a fixed opening located in a middle of the frame, in which the frame connects to the gas delivery hose through the fixed opening, and the fixed opening is a tubular body of a certain length, providing sufficient contact surface for the connection of the gas delivery hose to the fixed opening;
first connection parts located on two sides of the frame in a same horizontal direction, wherein the frame connects to the side connectors through the first connection parts, and the gas delivery hose is configured to rotate relative to the frame when the gas delivery hose is connected to the frame.

The gas delivery hose includes:
a first end located at an upper part of the gas delivery hose, in which the first end is configured for detachable connection to the frame and the first end has an exhaust channel to allow airflow from the gas delivery hose to an external environment;
a middle hose positioned between the first end and a second end to connect with the first end and the second end, wherein the middle hose has multiple continuous evenly folded walls that allow for bending and stretching of the middle hose;
the second end located at a lower part of the gas delivery hose and configured to connect with an other tubular connector; the second end consisting of a first part and a second part, in which the first part is fixedly connected to a lower end of the middle hose, with an other end of the first part away from the middle hose connected to the second part, and an other end of the second part connects with the other tubular connector, thereby being configured to deliver breathable gas to the gas delivery hose, which is then channeled through the frame and the nasal pillow to the user's nostrils.

The side connectors are provided in a pair, connecting to the first connection parts on both sides of the frame; and the side connectors have a front end and a rear end, the front end connecting to the frame, and the rear end is configured to make contact with the user's face.

The frame assembly directly connected to a frame and a short tube further includes: an exhaust channel which is located on the first end of the gas delivery hose, and configured allow the gas exhaled from the user's nose to be directed to the nasal pillow when the nasal pillow is used. Then the gas flows from the nasal pillow through the frame into the gas delivery hose. And the exhaled gas is eventually expelled through the exhaust channel on the first end of the gas delivery hose.

In an embodiment, the fixed opening has a central axis located in the middle of the fixed opening, the frame is a symmetrical shape with left-right symmetry along a straight line which is the axis of symmetry for the frame, and the central axis and the axis of symmetry have at least one of the following characteristics: A. An angle α, formed by the central axis of the fixed opening and the straight line connecting the furthest point on the frame from the fixed opening and the center point defined by the central axis of the fixed opening, is at or between 0° to 90° towards the user's face; B. The angle β, formed by the axis of symmetry of the frame and the central axis of the fixed opening, is at or between 30° and 150°.

In an embodiment, a length of the gas delivery hose is at or between 200 to 600 mm, an external diameter of the gas delivery hose is between 25 to 45 mm, and a wall thickness of the gas delivery hoses is at or between 1 to 5 mm, with an expandability of the gas delivery hose being at or between 80% to 120%.

In an embodiment, the connections between the gas delivery hose and the frame, the side connectors and the frame, and the nasal pillow and the frame includes one or more of buckles, snap fasteners, hook and loop fasteners, rotary knobs, or magnetic attraction fasteners.

In an embodiment, there are multiple exhaust channels on the first end of the gas delivery hose, and a combined area of the exhaust channels constitutes 20%-50% of an outer surface area of the first end of the gas delivery hose.

In an embodiment, the exhaust channel is positioned towards a side of the gas delivery hose, includes a noise reduction piece that covers the exhaust channel and is configured to reduce noise when the gas delivery hose vents to the external environment through the exhaust channel.

In an embodiment, the exhaust channel includes exhaust grids or exhaust holes, and the exhaust grids have through holes opened on a surface of the gas delivery hose and are attached to the gas delivery hose to cover the through holes by adhesive, buttons, ultrasonic bonding, or heat pressing; and the exhaust holes are formed by multiple small circular holes on the surface of the gas delivery hose.

In an embodiment, the second part of the second end of the gas delivery hose is a cylinder that is configured to rotate coaxially relative to the first part, allowing the second part to achieve coaxial rotation relative to the gas delivery hose.

In an embodiment, the front end and rear end of the side connectors are integrally formed through injection molding, and a linear distance between a center point of the front end and a center point of the rear end is at or between 20 to 200 mm.

In an embodiment, the noise reduction piece includes one or more of noise-reducing cotton or noise-reducing mesh, and the noise-reducing cotton is made from at least one material selected from polyester, polypropylene, polyethylene, nylon, vinylon, and natural fabric; and the noise-reducing mesh is made from at least one material selected from polyvinyl chloride, polypropylene, polytetrafluoroethylene, or nylon.

In an embodiment, a frame assembly directly connected to a frame and a short tube is provided. A frame assembly directly connected to a frame and a short tube, comprises a frame for securing a nasal pillow, a gas delivery hose connected to the frame for delivering breathable gas to the nasal pillow, and side connectors for securing the frame to a user's face. The frame is configured to accommodate the nasal pillow and position the nasal pillow at the user's nostrils with at least a part of the frame being rigid, and the frame is configured to bend towards the user's face such that the frame fits to the user's face and ensures a secure attachment of the nasal pillow to the user's face.

The frame includes:
a fixed opening located in a middle of the frame, in which the frame connects to the gas delivery hose through the fixed opening, and the fixed opening is a tubular body of a certain length, providing sufficient contact surface for the connection of the gas delivery hose to the fixed opening;
first connection parts located on two sides of the frame in a same horizontal direction, in which the frame connects to the side connectors through the first connection parts.

The gas delivery hose includes:
a first end located at an upper part of the gas delivery hose for detachable connection to the frame, in which the gas delivery hose is configured to rotate relative to the frame once the gas delivery hose is connected to the frame;
a middle hose positioned between the first end and a second end to connect with the first end and the second end, in which the middle hose has multiple continuous evenly folded walls that allow for bending and stretching of the middle hose;
the second end located at a lower part of the gas delivery hose and configured to connect with an other tubular connector; the second end consisting of a first part and a second part, in which the first part is fixedly connected to a lower end of the middle hose, with an other end of the first part away from the middle hose connected to the second part, and an other end of the second part connects with the other tubular connector, thereby being configured to deliver breathable gas to the gas delivery hose, which is then channeled through the frame and the nasal pillow to the user's nostrils.

The side connectors are connected to the first connection parts on both sides of the frame; and the side connectors have a front end and a rear end, the front end connecting to the frame, and the rear end is configured to make contact with the user's face.

The frame assembly directly connected to a frame and a short tube further includes: an exhaust channel and an exhaust connector.

The exhaust channel is located on the exhaust connector, and is configured to allow a user to exhale gas from the user's nostrils to the nasal pillow, then exhaled gas flows sequentially through the nasal pillow to the frame and the exhaust connector, finally being vented out through the exhaust channel on the exhaust connector.

The exhaust connector includes a first connector and a second connector. The first connector is configured to connect with the fixed opening of the frame. And the second connector is configured to connect with the first end of the gas delivery hose.

In an embodiment, the fixed opening has a central axis located in the middle of the fixed opening. The frame is a symmetrical shape with left-right symmetry along a straight line which is the axis of symmetry for the frame. The central axis and the axis of symmetry have at least one of the following characteristics: A. An angle α, formed by the central axis of the fixed opening and the straight line connecting the furthest point on the frame from the fixed opening and the center point defined by the central axis of the fixed opening, is at or between 0° to 90° towards the user's face; B. The angle β, formed by the axis of symmetry of the frame and the central axis of the fixed opening, is at or between 30° and 150°.

In an embodiment, a length of the gas delivery hose is at or between 200 to 600 mm. An external diameter of the gas delivery hose is between 25 to 45 mm. And a wall thickness of the gas delivery hoses is at or between 1 to 5 mm, with an expandability of the gas delivery hose being at or between 80% to 120%.

In an embodiment, the connections between the gas delivery hose and the frame, the side connectors and the frame, and the nasal pillow and the frame includes one or more of buckles, snap fasteners, hook and loop fasteners, rotary knobs, or magnetic attraction fasteners.

In an embodiment, there are multiple exhaust channels on the exhaust connector, and a combined area of the exhaust channels constitutes at or between 20% to 50% of an outer surface area of the exhaust connector.

In an embodiment, the exhaust channel has a noise reduction piece which covers an exhaust position of the exhaust channel to reduce noise when the exhaust connector vents to an external environment through the exhaust channel.

In an embodiment, the exhaust channel includes exhaust grids or exhaust holes. The exhaust grids are made of through holes opened on a surface of the exhaust connector and are attached to the exhaust connector to cover the through holes by adhesive, buttons, ultrasonic bonding, or heat pressing; and the exhaust holes are formed by multiple small circular holes on the surface of the exhaust connector.

In an embodiment, the second part of the second end of the gas delivery hose is a cylinder that is configured to rotate coaxially relative to the first part, allowing the second part to achieve coaxial rotation relative to the gas delivery hose.

In an embodiment, the front end and rear end of the side connectors are integrally formed through injection molding. A linear distance between a center point of the front end and a center point of the rear end is at or between 20 to 200 mm.

In an embodiment, the noise reduction piece includes one or more of noise-reducing cotton or noise-reducing mesh. The noise-reducing cotton is made from at least one material selected from polyester, polypropylene, polyethylene, nylon, vinylon, and natural fabric; and the noise-reducing mesh is made from at least one material selected from polyvinyl chloride, polypropylene, polytetrafluoroethylene, or nylon.

Method to implement the frame assembly directly connected to a frame and a short tube provided by this disclosure is as follows. First, the nasal pillow is connected to a side of the frame opposite the fixed opening. The first end of the gas delivery hose connected to the fixed opening of the frame is held with one hand to position the frame towards the side of the face, aligning with the use's nose, which ensures the nasal pillow seals the user's nose so as to provide pressurized or breathable gas. After the nose is sealed by the nasal pillow, another hand is used to secure the two side connectors of the first connection parts of the frame to the user's head to establish the connection between the frame and the user. The nasal pillow is secured at the user's nose to enhance stability when using the nasal pillow. The fixed opening of the frame is connected to the gas delivery hose, and the first end of the gas delivery hose is connected to the frame through the fixed opening. The middle of the gas delivery hose features a middle hose with multiple evenly spaced, collapsible walls. This gives the middle hose elasticity, allowing it to rotate relative to the frame, ensuring flexible use for the user. The second end of the gas delivery hose is meant to connect with other tubular connectors, providing breathable gas within the hose to satisfy the user's regular breathing needs. The frame assembly also have an exhaust channel. The exhaust channel can be situated on the frame, the first end, or on the exhaust connector between the first end and the fixed opening. The exhaust channel can be configured in the form of exhaust grids or exhaust holes paired with noise-reducing pieces. Through the configuration of the exhaust channel, exhaled gas from the user is promptly released, facilitating the user's intake of fresh air. By placing the exhaust channel on the frame or the first end, the exhaust function of the frame assembly can be achieved without the need for additional devices or equipment, e.g., without an elbow that are required in traditional devices. This can reduce excess parts, lightens the overall weight of the frame assembly, more convenient to use. When worn on the user's head, it also enhances the user experience.

Implementing the frame assembly directly connected to a frame and a short tube of this disclosure has several beneficial effects, including but not limited to:

1. Reduce parts and weight. Most mask systems in the market connect the patient interface pad to other tubular connectors through a combination of the frame, elbow, conduit, and straps, forming a complete gas delivery channel, and delivers pressurized gas to the patient's or user's airway, achieving the purpose of preventing sleep apnea. When wearing the mask system for extended periods of time at night, the weight of the system may press down on the user, leading to discomfort for some users and causing impressions on the skin, psychological stress, and other sleep disturbances. Therefore, the device as discussed herein innovatively constructs the frame assembly using only three parts—the frame, the gas delivery hose, and side straps. Combined with the redesigned nasal pillow, a completely new nasal mask system is developed. In the new frame assembly, the frame is designed to connect directly with the gas delivery hose without an elbow. That is, the connection of the exhaust channel on the elbow and the frame is redesigned, eliminating the traditional elbow part and configuring an exhaust channel on the frame which can be combined with noise-reducing materials. This not only reduces the noise of the product, ensuring a quiet environment for users during use but also lightens the overall weight of the assembly, making it more comfortable to wear, thereby enhancing the comfort and overall experience of the user.

2. Simplify user operation. The connection between the gas delivery hose and the frame is configured to be detachable, streamlining the installation process. This allows users to quickly disassemble and wear the mask system, preventing scenarios where users need to move quickly, such as waking up at night or in emergencies, but are not able to promptly separate the mask from the machine. Also, the second end of the gas delivery hose can rotate 360°. Compared to the current permanent connection method between the gas delivery hose and the frame, the detachable and rotatable design reduces the likelihood of the hose getting tangled due to being fixed at both ends. This greatly enhances the flexibility and adaptability of the frame assembly. Furthermore, the detachable hose offers users a more convenient option for cleaning and replacement. Users can carry out regular maintenance and cleaning, ensuring the mask system remains in good condition and avoiding potential health risks.

3. More economical and environmentally friendly for users. The application of the detachable gas delivery hose brings numerous economic and environmental advantages. Given the vulnerability of the hose material compared to other parts of the frame assembly, this detachable design allows users to replace only the damaged part, such as the gas delivery hose or other parts, without replacing the entire frame assembly. This reduces operational and maintenance costs and prolongs the overall system's lifespan. It enhances the durability and reusability of the gas delivery hose, making maintenance and repair of the entire frame assembly more efficient, cost-effective, and environmentally friendly.

4. Ease of Manufacturing for Producers. From the production perspective, the use of the detachable connection structure for the assembly makes the manufacturing process more convenient, avoiding the common issues of damage in traditional welding or fixed connections, which is often encountered in the production of integrated frame assemblies and gas delivery hoses. As a result, the defect rate is significantly reduced. When maintenance is required or damaged components need to be replaced, only simple mechanical disassembly is needed. This process allows for quick inspection and repair, ensuring the continuity and stability of the production process. Through such straightforward mechanical structures, the efficiency of the production line is maintained, providing a more efficient and reliable production process for manufacturers.

5. Enhanced comfort in use. To improve comfort when wearing, special attention is given to the design of the side connectors. To adapt to facial deformities, a deformable material with inherent support properties selected and an integrated injection molding process is used to prevent any joint marks that can cause discomfort on the face. The overall side connector has a smooth arc, fitting more closely to the user's facial deformities, offering a more comfortable wearing experience. Multiple connection options are provided, including one or more of buckles, snap fasteners, hook and loop fasteners, rotary knobs, and magnetic attraction fasteners, for flexible connection with the frame. To further enhance the device's usability, “L/R” embossed signs, indicating left and right, are marked on the side connectors, assisting users to quickly and easily wear the mask even in dark environments, providing a more intelligent and convenient user experience.

In summary, the device as discussed herein innovatively constructs the frame assembly as a combination of only three parts: the frame, the gas delivery hose, and the side connectors. While retaining the device's primary functions, it achieves an enhanced user experience, improving the overall comfort of the device, making it easier to clean, and significantly extending its overall lifespan. Moreover, the detachable design effectively reduces production costs and actively contributes to environmental protection. The disassembling structure combination and manufacturing process make the production process more efficient and economical, minimizing resource waste. Additionally, its detachable and reusable features contribute to reducing waste, making it more environmentally friendly. Overall, the design of this frame assembly directly connected to a frame and a short tube not only enhances functionality and user experience but also balances comfort and environmental considerations, offering users a more convenient, efficient, and sustainable user experience.

### BRIEF DESCRIPTION OF THE DRAWINGS

FIG. 1 is an overall schematic diagram of the frame assembly directly connected to a frame and a short tube provided in accordance with an example embodiment;

FIG. 2 is an exploded schematic diagram illustrating the structural breakdown of the gas delivery hose of the frame assembly directly connected to a frame and a short tube in accordance with an example embodiment;

FIG. 3 is a schematic diagram showcasing the exhaust grids designed on the frame of the frame assembly directly connected to a frame and a short tube in accordance with an example embodiment;

FIG. 4 is a schematic diagram showcasing the exhaust holes designed on the frame of the frame assembly directly connected to a frame and a short tube in accordance with an example embodiment;

FIG. 5 is a schematic diagram depicting the frame in the frame assembly directly connected to a frame and a short tube, incorporating both an exhaust hole and noise-reducing pieces in accordance with an example embodiment;

FIG. 6 is a schematic diagram illustrating the first end of the frame assembly directly connected to a frame and a short tube, configured with exhaust grids in accordance with an example embodiment;

FIG. 7 is a schematic diagram illustrating the first end of the frame assembly directly connected to a frame and a short tube, configured with exhaust holes in accordance with an example embodiment;

FIG. 8 is a schematic diagram illustrating the first end of the frame assembly directly connected to a frame and a short tube, configured with exhaust holes and noise-reducing pieces in accordance with an example embodiment;

FIGS. 9A and 9B are combined schematic diagrams representing the frame assembly directly connected to a frame and a short tube that integrates the exhaust connector in accordance with an example embodiment;

FIG. 10 is an exploded schematic diagram of the frame assembly directly connected to a frame and a short tube, focusing on the exhaust connector feature in accordance with an example embodiment;

FIG. 11 is a schematic diagram illustrating the angle α of the frame assembly directly connected to a frame and a short tube in accordance with an example embodiment;

FIG. 12 is a schematic diagram showcasing the maximum angle α of the frame assembly directly connected to a frame and a short tube in accordance with an example embodiment;

FIG. 13 is a schematic diagram depicting the angle β of the frame assembly directly connected to a frame and a short tube in accordance with an example embodiment;

FIGS. 14A, 14B, and 14C are schematic usage diagrams representing the various angle β in the frame assembly directly connected to a frame and a short tube in accordance with an example embodiment;

FIGS. 15A, 15B, and 15C are schematic diagrams showcasing different connection methods between the first end of the gas delivery hose and the frame in the frame assembly directly connected to a frame and a short tube in accordance with an example embodiment;

FIG. 16 is a schematic diagram illustrating a magnetic connection between the first end of the gas delivery hose and the frame in the frame assembly directly connected to a frame and a short tube in accordance with an example embodiment;

FIG. 17 is a schematic diagram showing a buckle connection between the first end of the gas delivery hose and the frame in the frame assembly directly connected to a frame and a short tube in accordance with an example embodiment;

FIG. 18 is a schematic diagram illustrating a magnetic connection between the side connector and the frame in the frame assembly directly connected to a frame and a short tube in accordance with an example embodiment;

FIG. 19 is a schematic diagram detailing a buckle connection between the side connector and the frame in the frame assembly directly connected to a frame and a short tube in accordance with an example embodiment;

FIGS. 20A and 20B are schematic diagrams of the structure of the side connector in the frame assembly directly connected to a frame and a short tube in accordance with an example embodiment;

FIGS. 21A and 21B are schematic diagrams showing different orientations of the fixed opening in the frame assembly directly connected to a frame and a short tube in accordance with an example embodiment;

FIG. 22 is a schematic diagram illustrating the combination of the frame assembly directly connected to a frame and a short tube and the nasal pillow in accordance with an example embodiment;

FIG. 23 is a schematic usage diagram of the frame assembly directly connected to a frame and a short tube in accordance with an example embodiment.

Labeling is as follows:
1—frame; 11—Fixed opening; 2—Gas delivery hose; 21—Middle hose; 22—First end; 23—Second end; 231—First part; 232—Second part; 3—Side connector; 31—Front end; 32—Rear end; 4—Exhaust channel; 5—Exhaust connector; 51—First connector; 52—Second connector; 6—Nasal pillow; 7—Noise-reducing piece.

### DETAILED DESCRIPTION

To facilitate an understanding of this disclosure, a more comprehensive description will be provided below with reference to the relevant drawings. The drawings present typical embodiments of this disclosure. However, it should be understood that the disclosure can be implemented in many different forms and is not limited to the embodiments described herein. Rather, these embodiments are provided to make the disclosure more thorough and comprehensive.

Unless otherwise defined, all technical and scientific terms used herein have the same meaning as commonly understood by those of ordinary skill in the art to which this disclosure belongs. The terms used in the description of the disclosure herein are for the purpose of describing particular embodiments only and are not intended to be limiting of the disclosure.

### Embodiment 1

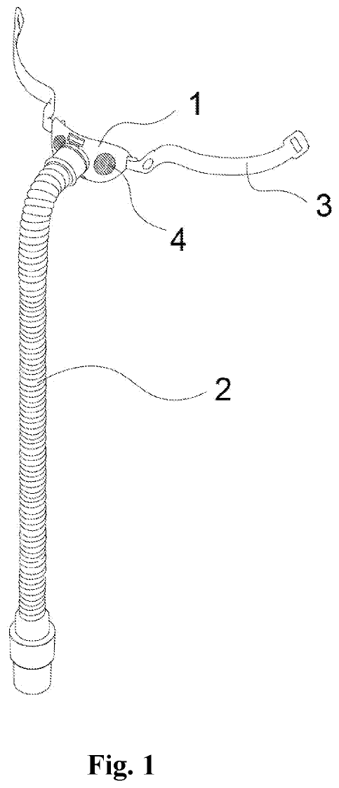

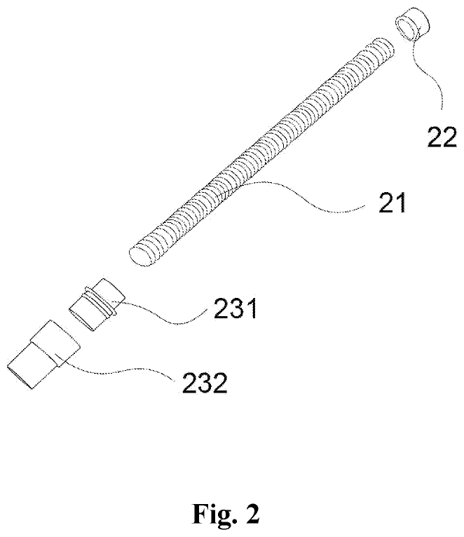

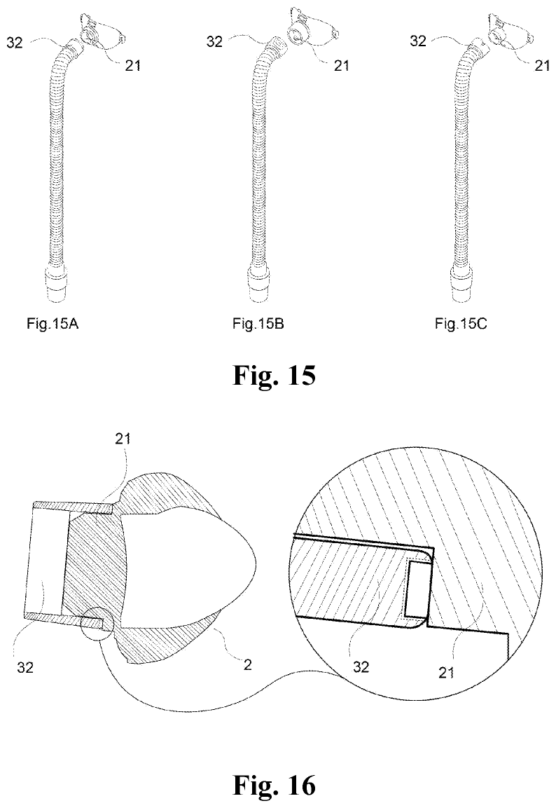

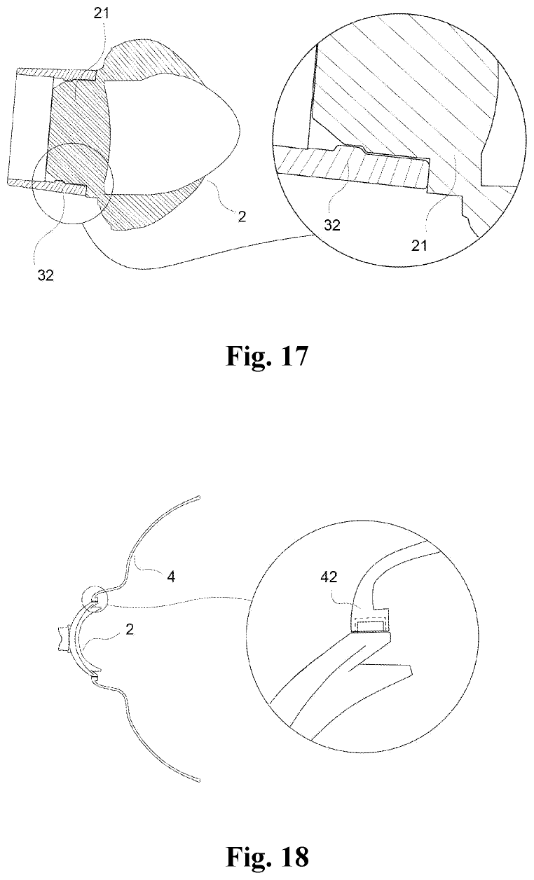

This embodiment provides a frame assembly directly connected to a frame and a short tube. Referring to FIG. 1, the frame assembly directly connected to a frame and a short tube includes a frame 1 for securing a nasal pillow 6, a gas delivery hose 2 for conveying breathable gas, and side connectors 3 for affixing the frame 1 to the user's face. At least a part of the frame 1 is rigid. And the frame can be made from medical-grade plastic materials such as polyethylene, polypropylene, polycarbonate, polymethyl 1 methacrylate, polyvinyl chloride, and acrylonitrile butadiene phenethylester, to accommodate the nasal pillow 6 and position it at the user's nostrils, allowing the nasal pillow 6 to fit with the user's face in a fixed state. The nasal pillow 6 is detachably connected to the frame 1. Specifically, the nasal pillow 6 can be attached to the frame 1 by various means such as buckles, snap fasteners, hook and loop fasteners, rotary knobs, or magnetic attraction fasteners. The frame 1 is configured to bend towards the user's face (i.e. having a contour that roughly conforms to the shape of the face) to allow the frame 1 to fit with the user's face and to ensure a secure attachment between the nasal pillow 6 to the face. When the frame 1 is secured to the user's face, it ensures the nasal pillow 6 is sealed stably around the user's nose, facilitating the steady delivery of breathable gas to the user's nostrils, thus ensuring normal respiration. The frame 1 has the fixed opening 11 for connection to the gas delivery hose 2. The fixed opening 11 is situated on the frame 1. The frame 1 communicates with the gas delivery hose 2 through the fixed opening 11, ensuring that the gas in the gas delivery hose 2 is delivered through a connection of the gas delivery hose 2 with the frame 1 to the nasal pillow 6 and then delivered to the user's nose. The fixed opening 11 is tubular with a certain length, providing sufficient contact surface (at least 30 mm²) for the connection of the gas delivery hose 2 and the frame 1, thus securing the connection of the frame 1 and the gas delivery hose 2. Specifically, the wall thickness of the fixed opening 11 is at or between 0.3 to 5 mm; the length of the fixed opening 11 is at or between 0.3 to 30 mm. Depending on various scenarios, different sizes of the fixed opening 11 can be selected to meet a wide array of user requirements, enhancing the applicability range of the frame assembly. The total length of the gas delivery hose 2 is at or between 200 to 600 millimeters and is configured to convey breathable gas to the user. The manufacturing materials for the gas delivery hose 2 include but are not limited to one of plastic materials, silicone, thermoplastic elastomers, and silicone resin. The gas delivery hose 2 includes a first end 22 that connects with the frame 1 through the fixed opening 11, and a second end 23 that connects to another tubular connector as shown in FIG. 2. The first end 22 and the second end 23 are interconnected by a middle hose 21. The middle hose 21 is hollow in its center, having several multiple continuous evenly folded walls, allowing for compression and extension of the middle hole relative to the first end 22 and the second end 23. The external diameter of the gas delivery hose 2 is at or between 25 to 45 mm. The wall thickness of the gas delivery hose 2 is at or between 1 to 5 mm. The gas delivery hose 2 can expand and contract at or between 80% to 120%. This allows the second end 23 of the gas delivery hose 2 to stretch and compress relative to the first end 22, and allows the user to move within a certain range while wearing the frame assembly. To simplify the internal structure and reduce the overall weight, the first end 22 of the gas delivery hose 2 is approximately cylindrical. Consequently, the cross-sectional shape of the fixed opening 11 is also circular. In other embodiments, the cross-section of the gas delivery hose 2 could be in other shapes such as elliptical or polygonal, with the fixed opening 11 adopting a shape corresponding to the gas delivery hose 2 to facilitate the connection. The length of the fixed opening 11 is at or between 10 to 25 millimeters; the external diameter of the fixed opening 11 is at or between 15 to 25 millimeters; the wall thickness of the fixed opening is at or between 1 to 3 millimeters. To ensure sufficient gripping area for the user during operation e.g., having a length for the user to grab, and to avoid adding unnecessary weight, the length of the first end 22 is at or between 10 to 25 millimeters, weighing approximately at or between 1 to 10 grams. The first end 22 connects to the fixed opening 11 in a detachable manner. They can be connected through any methods from buckles, snap fasteners, friction fit, hook and loop fasteners, rotary knobs, or magnetic attraction fasteners. FIGS. 15 to 17 illustrate four types of connections: buckles, snap fasteners, friction fit, and magnetic attraction fasteners. In FIG. 15A, the first end 22 connects to the frame 1 by the buckle, and the first end 22 is a plastic material with certain resilience. The inner wall of the first end 22 is configured with protrusion, and the outer wall of the fixed opening 11 is configured with the corresponding recess for the protrusion. This design allows the first end 22 to be sleeved over the fixed opening 11. In other embodiments, the fixed opening 11 can be sleeved over the outer side of the first end 22. That is, the first end 22 is configured with the recess, and the fixed opening 11 is configured with the corresponding protrusion, enabling the detachable connection between the gas delivery hose 2 and the frame 1. The specific connection method can be found in FIG. 17. FIG. 15B illustrates the first end 22 connecting to the frame 1 through the snap fasteners. Specifically, the first end 22 is a plastic material with certain resilience, and the inner wall of the fixed opening 11 is configured with the elastic part. By pushing the first end 22 into the fixed opening 11, the elastic part inside the fixed opening 11 tightly connects with the outer wall of the first end 22. Similarly, in other embodiments, the connection method in which the first end 22 full fits over the fixed opening 11 can also be adopted. That is, the fixed opening 11 is a plastic material with certain resilience, and the inner wall of the first end 22 is configured with the elastic part. By pushing the first end 22 into the fixed opening 11, a tight connection is achieved between the elastic part inside the fixed opening 11 and the outer wall of the first end 22. In FIG. 15C, the first end 22, which is the elastic part, is directly deformed to be pushed into the fixed opening 11, utilizing the friction from its elasticity to connect with the outer wall of the frame 1. The material of the first end 22 can differ from that of the frame, such as rubber, silicone, or thermoplastic elastomers, like silicone material with a hardness of 30-70 Shore A or TPE with a hardness of 20-80 Shore A. The materials mentioned above are neither too soft nor too hard, providing a good sealing effect, wear resistance, and can deform to connect with the fixed opening 11. FIG. 16 illustrates the magnetic connection between the first end 22 and the fixed opening 11. Both the first end 22 and the fixed opening 11 are configured with magnets embedded, allowing them to connect through magnetic attraction fasteners. The second end 23 consists of a first part 231 and a second part 232. The first part 231 is a cylindrical tube fixedly connected to the second end 23, while the second part 232 connects to the end of the first part 231 that is furthest from the middle hose 21 and can rotate coaxially relative to the first part 231, achieving coaxial rotation of the second part 232 relative to the middle hose 21. When the first end 22 of the gas delivery hose 2 connects with the fixed opening 11, the first end 22 of the gas delivery hose 2 forms a rotatable connection with the frame 1, enabling a 360-degree rotation of the gas delivery hose 2 relative to the frame 1. The first end 22 is fixedly connected to the middle hose 21, while the second part 232 of the second end 23 can rotate coaxially relative to the middle hose 21, and thus the second end 23 can rotate coaxially relative to the first end 22 and the frame 1, avoiding that the gas delivery hose 2 get tangled during use. The second part 232 is configured to connect with other tubular connectors that have a similar cross-sectional interface, allowing gas to enter the gas delivery hose 2 from the second end 23. Specifically, the overall shape of the second end 23 is cylindrical corresponding to the first end 22. The length of the second end is approximately at or between 45 to 55 millimeters; the external diameter of the second end is approximately at or between of 15 to 25 millimeters; the wall thickness of the second end is approximately at or between 1 to 3 millimeters. The second end can be made of elastic materials like plastic or silicone to facilitate a better connection with external tubular connectors.

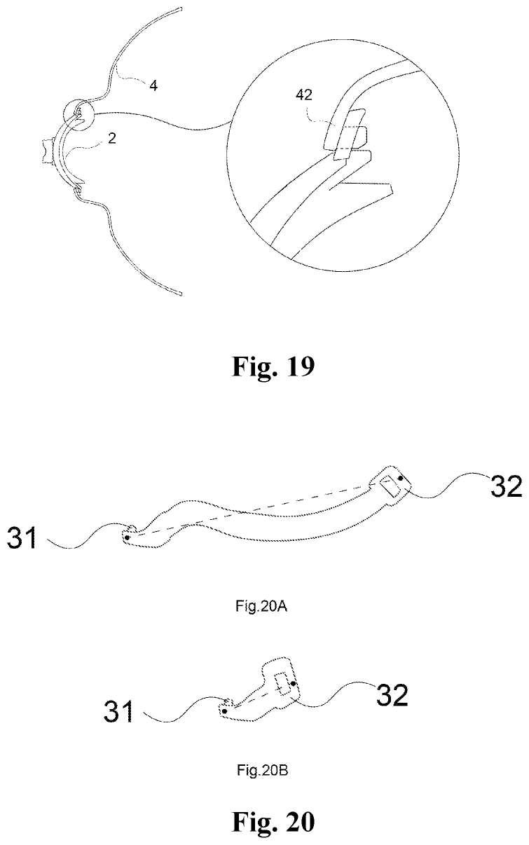

The frame is a symmetrical shape with left-right symmetry along a straight line, with first connection parts positioned on both sides of the frame 1 in a horizontal direction (perpendicular to the symmetrical axis of the frame in the left-right direction). In other words, the first connection parts are located on both the left and right sides of the frame 1. The two ends of the frame 1 are configured with the first connection part connected to side connector 3. The side connector 3 has a front end 31 connected to the frame 1 and a corresponding rear end 32. The front end 31 is detachably connected to the frame 1. Specifically, it can be connected by any method from buckle as shown in FIG. 19, snap fasteners, hook and loop fasteners, rotary knobs, or magnetic attraction fasteners as shown in FIG. 18. In an embodiment, only snap fasteners and magnetic attraction fasteners are shown, that is, magnets or connection buckles are set at both ends of the connection between the side connector 3 and the frame 1, achieving a detachable connection between the side connector 3 and the frame 1.

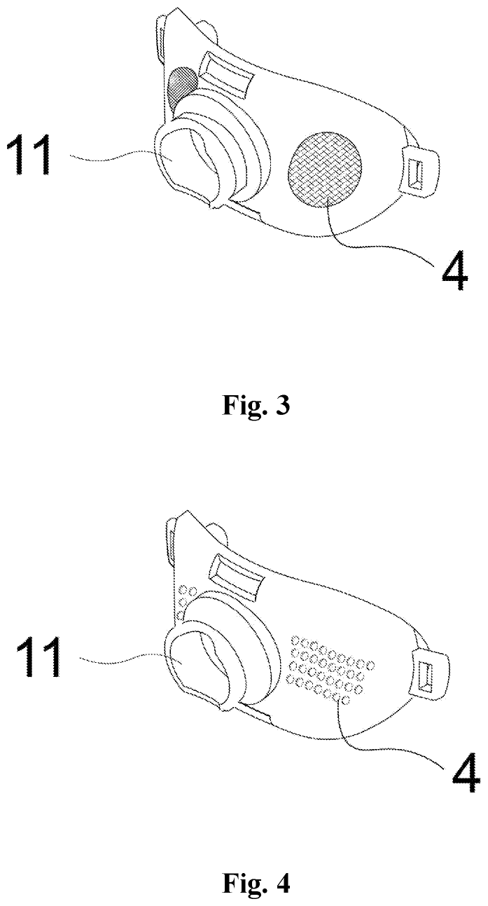

The side connector includes side straps and side buckles. The frame assembly has two side connectors 3, which, for production convenience, are symmetrical and of the same size, respectively fitting on the left and right sides of the user's face. To better fit the side connectors 3 to the face, the side connectors 3 is designed in a continuous and smooth arc shape with a width of approximately at or between 5 to 25 millimeters and a linear distance from the point on the front end 31 of the side connector 3 to the point on the rear end 32 being at or between 20 to 200 millimeters as shown in FIG. 20. The side connectors 3 partly fit the face. Taking into account the linear distance from the tip of the user's nose to the tip of the ear, combined with the distance between frame 1 and the user's face, the linear distance from the point on the front end 31 to the point on the rear end 32 of the side connector 3 is at or between 40 to 120 millimeters; the wall thickness of the side connectors is approximately at or between 1 to 2 millimeters. To improve production efficiency, the side connector 3 is integrally injection molded. Through structural characteristics such as wall thickness, length, and shape, the side connectors 3 more readily deforms to fit the user's face, enhancing the user experience. The frame assembly also is configured with an exhaust channel 4. In an embodiment, the exhaust channel 4 is located on frame 1 and positioned on two sides of the fixed opening 11. The exhaust channel 4 on both sides of the fixed opening 11 can be set symmetrically or asymmetrically. The shape of the exhaust channel 4 can be any symmetric or asymmetric shape, such as circular, semi-circular, elliptical, rectangular, square, etc. The venting area covers at or between 20% to 50% of the surface area of the frame 1. Under room temperature and an air humidity of 75% to 85%, when the positive pressure gas flows through the first end 22 of the gas delivery hose 2, the wind speed is at or between 2.25 to 2.45 m/s. When the venting area and the first end 22 of the gas delivery hose 2 together occupy about 20% of the surface area, the wind speed tested in the exhaust channel 4 of the gas delivery hose 2 is at or between 1.2 to 1.3 m/s, meeting the regular ventilation requirements and preventing the situation of insufficient gas flow. When the venting area and the first end 22 of the gas delivery hose 2 together occupy about 50% of the surface area, the wind speed tested in the exhaust channel 4 of the gas delivery hose 2 is at or between 1.6 to 1.7 m/s, meeting the positive pressure ventilation requirements. The exhaust channel 4 can be any of the following: the exhaust grids, the exhaust holes, or a combination of exhaust holes with the noise-reducing pieces 7. When the exhaust channel 4 are exhaust grids as shown in FIG. 3, it can be made of materials like polyvinyl chloride, polypropylene, polytetrafluoroethylene, or nylon. The exhaust mesh is securely attached and covers the through-holes on the frame 1 by means of adhesives, button, ultrasonic pressing, heat pressing, etc. to enhance the connection's tightness. When the exhaust channel 4 are exhaust holes as shown in FIG. 4, the exhaust holes are arranged in multiple small circular holes on the surface of frame 1; i.e. multiple uniform circular holes are made on the frame 1. The distribution and size of these holes are not fixed.

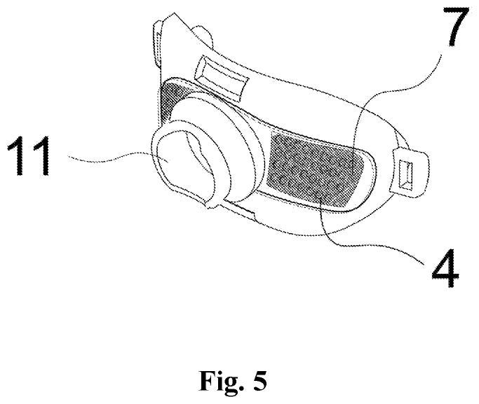

In other embodiments, square or triangular holes can also be adopted. The setting of the exhaust holes can improve the ease of setting up the exhaust channel 4, lighten the weight of the frame assembly while easily discharging exhaust gases, facilitating timely gas exchange for the user. As such, the exhaled gas finally exits via the exhaust channel on the frame, ensuring the user inhaling fresh air promptly. When the exhaust channel 4 combines exhaust holes with the noise-reducing pieces 7 as shown in FIG. 5, noise-reducing pieces 7 just are needed to be added at the exhaust holes. The noise-reducing pieces cover the outer surface of the exhaust holes, thus reducing the sound produced by exhaling. According to the testing method in ISO 17510:2015, when only the exhaust holes are used, the sound level can be at or between 40 to 50 decibels. After adding the noise-reducing pieces 7, the sound level can be at or between 19 to 30 decibels, reducing the noise by 10 to 30 decibels. The noise reduction of the frame assembly is effective. The noise-reducing pieces 7 can be one or more of noise-reducing cotton or mesh (composed of interwoven metallic, fibrous, or other flexible and extensible materials, with numerous interwoven threads forming spaced-apart openings). The noise-reducing cotton can be made from one of the materials: polyester, polypropylene, polyethylene, nylon, vinylon, or natural fabrics. The noise-reducing mesh can be made from one or more of the materials: polyvinyl chloride, polypropylene, polytetrafluoroethylene, or nylon. By setting the exhaust channel 4 on the frame 1, the exhaust function of the frame assembly can be achieved without adding new devices or equipment. Also setting the exhaust channel 4 on the frame 1 reduces extra assembly and lightens the overall weight of the frame assembly, thus more convenient to use. When worn on the user's head, it also enhances the user experience. When the frame assembly is in use, all its parts are connected for timely use, improving the efficiency of the frame assembly. Furthermore, the total weight of the frame assembly is at or between 20 to 25 grams, which is lighter than the existing technology, thus offering a better user experience.

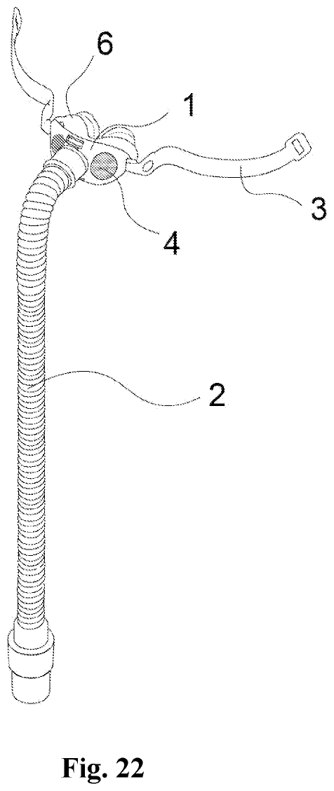

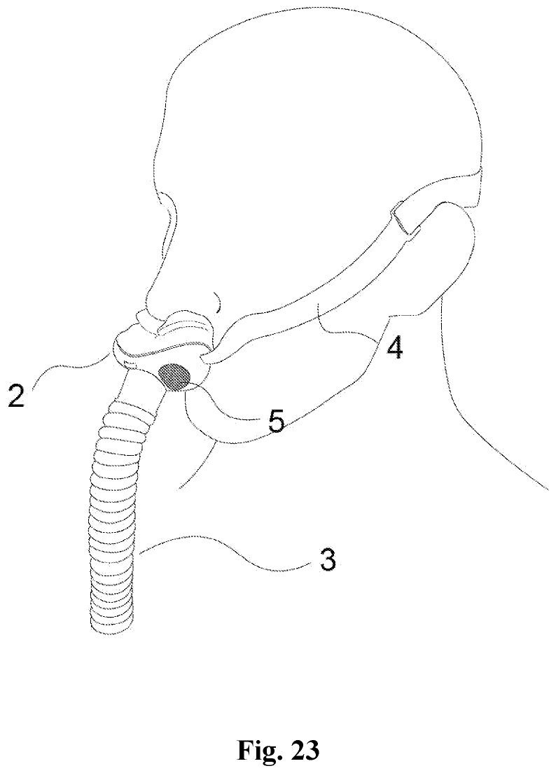

In the use of the frame assembly provided in an embodiment, the nasal pillow 6 is connected to the inside of the second connection parts of the frame 1 (as shown in FIG. 22). Hold the first end 22 of the gas delivery hose 2 connected to the fixed opening 11 of the frame 1 by one hand, and position the frame 1 towards the side of the face, aligning it with the user's nose, which ensures that the nasal pillow 6 seals the user's nose to provide pressurized or breathable gas. After sealing with the nasal pillow 6, fixing the two side connectors 3 of the first connection part of the frame 1 to the user's head with the other hand, thus establishing the connection between the frame 1 and the user. Secure the nasal pillow 6 at the user's nose (as shown in FIG. 23 to improve the stability during the use of the nasal pillow 6).

In summary, by eliminating the traditional elbow structure and directly connecting the frame 1 with the gas delivery hose 2, and integrating the exhaust channel 4 with the frame 1, not only noise of the product can be reduced when venting to the outside or external environment, ensuring a quiet environment for users during operation, but also the overall weight of the assembly can be lightened, making it more comfortable for users to wear and enhancing their comfort and user experience.

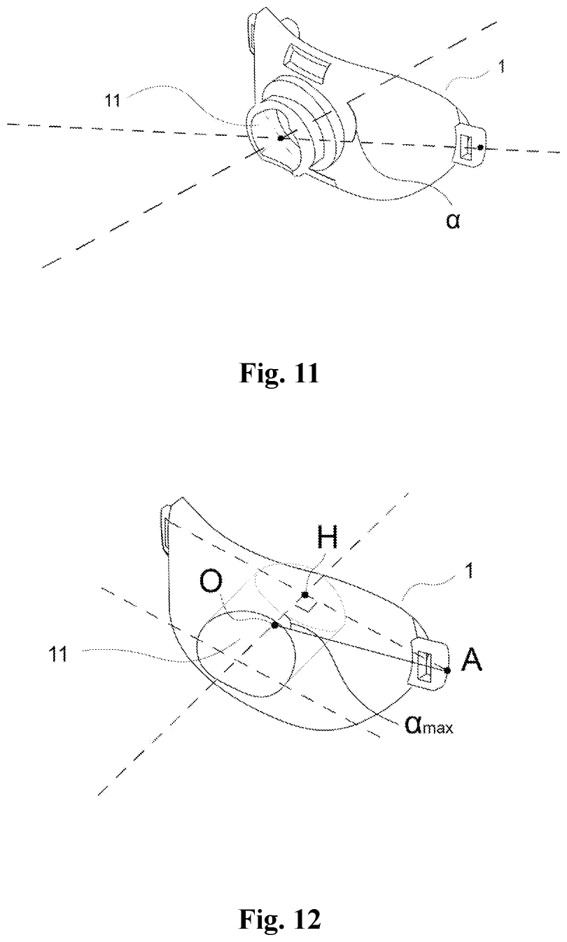

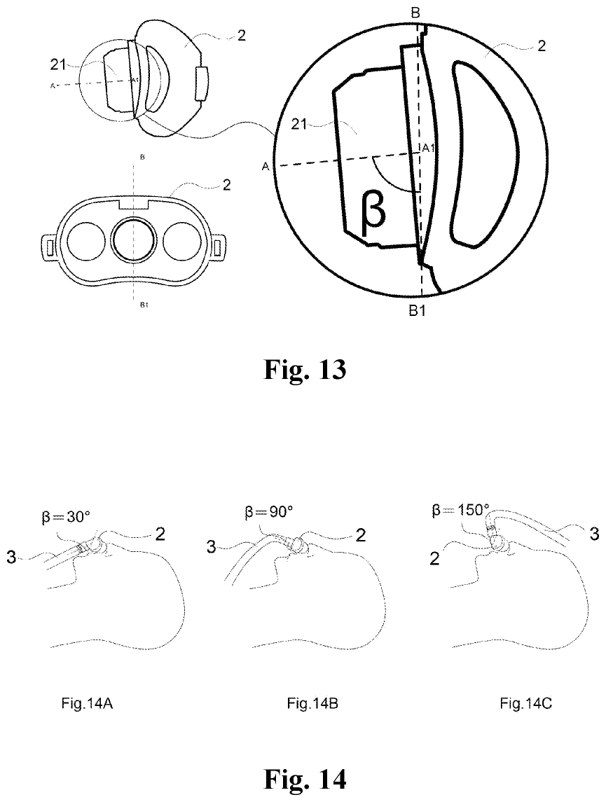

In another embodiment, the fixed opening 11 has at least one of the following characteristics: A. The angle α, formed by the central axis of the fixed opening 11 and the straight line connecting the furthest point on the frame from the fixed opening 11 and the center point of the fixed opening, is at or between 0° to 90° towards the face (as shown in FIG. 11); B. The angle β, formed by the axis of symmetry of the frame 1 and the central axis of the fixed opening 11, is at or between 30° and 150° (as shown in FIG. 13). Considering the comfort in use, the frame 1 is configured to bend toward the face. Regardless of the direction of the fixed opening 11, the length of the fixed opening 11 is limited by ergonomics, and the maximum extension towards the face does not exceed point A at the farthest end of the frame 1 or point H level with A. At this time, the center point O of the fixed opening 11 cannot coincide with H, and OH achieves the minimum. In the right-angled triangle HOA, the angle corresponding to OH increases gradually as point O moves outward, and the complementary angle α decreases correspondingly. Therefore, the range of angle α is at or between 0° to 90° (as shown in FIG. 13). Considering the durability of the product, the gas delivery hose 2, due to the specificity of its material, is more susceptible to damage than other assembly. If the connection angle between the gas delivery hose 2 and the frame 1 is appropriately designed, it can reduce the occurrence of damage due to stress and other issues. Different angles β can meet patients' various needs for placing the gas delivery hose 2 (as shown in FIG. 14), maintaining a suitable stress distribution of the gas delivery hose 2 in the frame 1, making it less likely to puncture. Therefore, the angle β, viewed from the front perspective of the frame 1, between the symmetry axis and the center axis of the fixed opening 11 is defined at or between 30° to 150°. FIG. 14 shows schematic diagrams of usage at different angles. The optimal range for β is at or between 30° to 90°. Specifically, when β is about 30° (as shown in FIG. 14A), the gas delivery hose 2 just touches the patient's chin. When β is about 150° (as shown in FIG. 14C), it can provide patients with an alternative placement of the gas delivery hose 2, placing it above the patient's face to avoid pulling the hose during frequent changes in sleeping postures at night. The selection of various angles can replace some functions of the elbow, providing patients with a comfortable therapeutic experience.

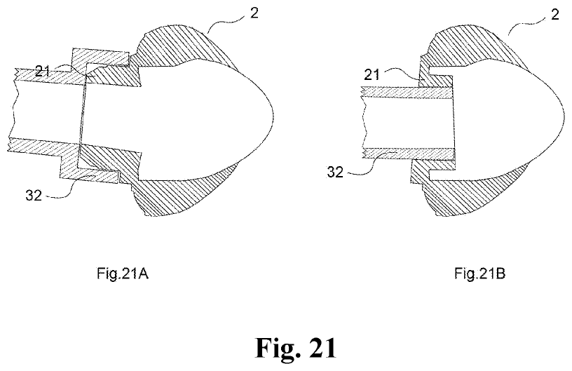

In another embodiment, catering to different users' preferences regarding the connection method between the gas delivery hose 2 and the frame 1, the fixed opening 11 features various orientations (as shown in FIG. 21B). The orientation can be set to face the side of the gas delivery hose 2 (illustrated on the left side of FIG. 21), namely protruding outward, or it can be oriented toward the side of the frame 1 (shown on the right side of FIG. 21), namely the fixed opening 11 is extending inward in the opposite direction. The first configuration exposes the fixed opening 11, facilitating a better connection between the fixed opening 11 and the first end 22 of the gas delivery hose 2, thereby enhancing the installation efficiency between the frame 1 and the gas delivery hose 2. The second configuration sets the tubular part of the fixed opening 11 within the cavity of the frame 1 to reduce the external exposure from the fixed opening 11, minimizing the space taken up by the fixed opening 11, thus improving the utility rate of space of the frame assembly. Depending on various requirements, both methods: inward setting and outward setting can accommodate more application scenarios, thereby broadening the frame assembly's applicability.

In another embodiment, to further enhance ease of use of the product, embossments of L/R are marked on the two side connectors 3, with L corresponding to the user's left side and R to the right. This addition of left-right indicators assists users in quickly donning the nasal pillow 6 even in dark environments, thereby improving the convenience of using the frame assembly and providing users with a more intelligent and convenient product experience.

### Embodiment 2

In an embodiment, a frame assembly directly connected to a frame and a short tube is provided. The difference of the embodiment 1 and the embodiment 2 lies in the setting position of the exhaust channel 4.

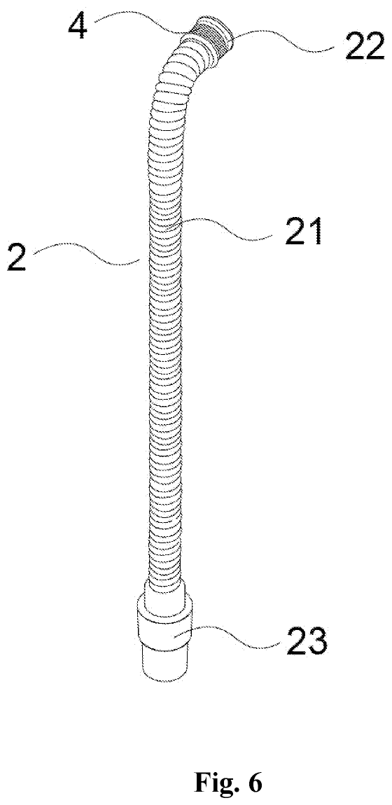

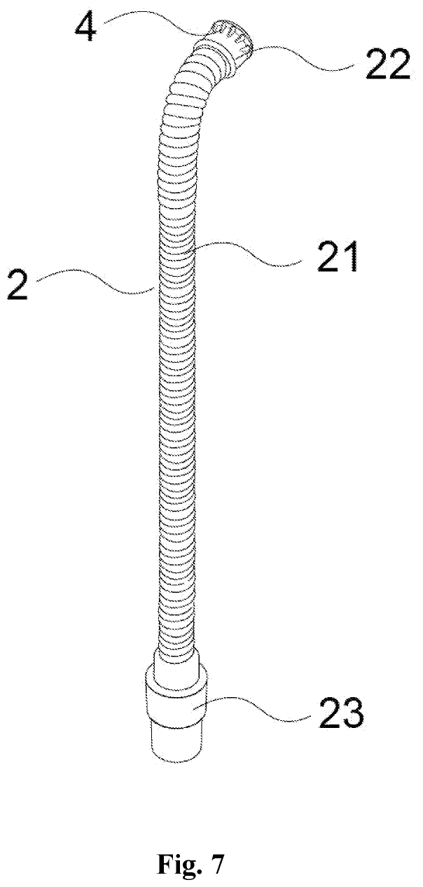

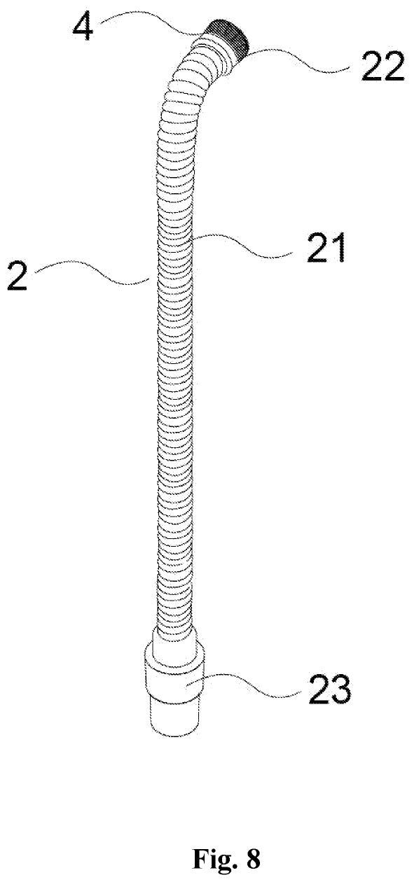

The frame assembly also includes the exhaust channel 4. in an embodiment, the exhaust channel 4 are located on the first end 22 of the gas delivery hose 2, specifically on the tubular side wall of the first end 22. The exhaled gas is expelled through the exhaust channel 4 on the first end 22 of the gas delivery hose 2. The shape of the exhaust channel 4 can be circular, semi-circular, elliptical, rectangular, square, or any other symmetrical or asymmetrical form. The ventilated area covers at or between 20% to 50% of the surface area of the first end 22. The exhaust channel 4 can be exhaust grids, exhaust holes, or a combination of exhaust holes with the noise-reducing pieces 7. When the exhaust channel 4 are exhaust grids (as shown in FIG. 6), the exhaust grids are covered around the side wall of the first end 22. The exhaust channel can be made from materials such as polyvinyl chloride, polypropylene, polytetrafluoroethylene, or nylon. The connection between the exhaust channel and the first end 22 can be directly and permanently secured by adhesives, ultrasonic pressing, heat pressing, etc., enhancing the tightness of the connection between the exhaust grids and the first end 22. When the exhaust channel 4 are exhaust holes (as shown in FIG. 7), droplet-shaped holes are evenly distributed at regular intervals on the first end 22 of the gas delivery hose 2. The orientation of the droplet-shaped exhaust holes aligns with the extension direction of the tubular body of the first end 22, with the larger caliber of the holes positioned closer to the fixed opening 11. The configuration of positioning the larger caliber closer to the fixed opening 11 shortens the distance that the gas travels from the fixed opening 11 to the exhaust channel 4, thereby improving the efficiency of waste gas discharge. The distribution and size of the exhaust holes are not fixed. In other embodiments, exhaust holes of different shapes, such as square or triangular, can be employed. The design of the exhaust holes enhances the convenience of establishing the exhaust channel 4 without adding the additional assembly, effortlessly facilitating waste gas expulsion while reducing the weight of the frame assembly and promoting timely gas exchange for the user. When the exhaust channel 4 are the combination of the exhaust holes and the noise-reducing pieces 7 (as shown in FIG. 8), the noise-reducing pieces 7 are simply added at the location of the exhaust holes. The noise-reducing pieces 7 cover the exhaust holes and encases the side wall of the first end 22, thereby diminishing the sound of the user's exhaled gas flow. According to tests conducted by methods in ISO 17510, the sound decibel level is at or between 40 to 50 decibels when using the exhaust holes alone. After adding the noise-reducing pieces 7, the sound decibel level drops to at or between 19 to 30 decibels, achieving a noise reduction at or between 10 to 30 decibels. The frame assembly assumes a good noise-reducing effect. The noise-reducing pieces 7 can be noise-reducing cotton or noise-reducing mesh. The noise-reducing cotton can be made from one of the materials: polyester, polypropylene, polyethylene, nylon, vinylon, or natural fabrics. The connection between the exhaust channel 4 and the first end 22 can be directly and permanently secured by adhesives, ultrasonic pressing, heat pressing, etc., or detachable methods like snap fasteners.

### Embodiment 3

In an embodiment, a frame assembly directly connected to a frame and a short tube is provided. The difference of the embodiment 3 and the embodiment 1 lies in the setting position of the exhaust channel 4.

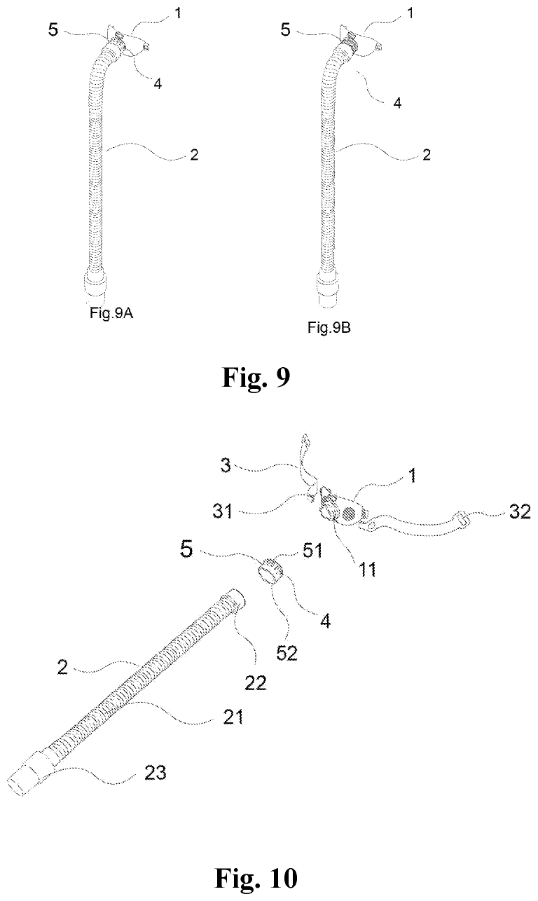

The frame assembly also includes the exhaust connector 5, which consist of a first connector 51 linked to the fixed opening 11 and a second connector 52 connected to the first end 22 of the gas delivery hose. The first connector 51 is detachably connected to the frame 1 via the fixed opening 11, while the second connector 52 is detachably linked with the gas delivery hose 2 through its first end 22 (as shown in FIG. 10). The cross-sectional profile of the exhaust connector 5 need to include at least one shape that matches the junction at the fixed opening 11 on the frame 1 and at least one shape that aligns with the junction at the first end 22 of the gas delivery hose 2. Parts of the exhaust connector 5 need to be able to slightly deform, and the materials include, but are not limited to, polypropylene, polycarbonate, polyethylene, silicone, and thermoplastic materials. The overall length of the exhaust connector 5 is at or between 10 to 25 mm, with an external diameter of at or between 20 to 28 mm, a wall thickness of at or between 1 to 3 mm, and a weight at or between 1 to 10 g. The exhaust channel 4 is located on the side wall of the exhaust connector 5 (as shown in FIG. 9). It allows a user to exhale gas from the user's nostrils to the nasal pillow 6, then the exhaled gas flows sequentially through the nasal pillow 6 to the frame 1 and the exhaust connector 5, and then vented out through the exhaust channel 4 on the exhaust connector 5. The ventilation area can occupy 20% to 50% of the surface area of the first end 22. The exhaust channel 4 can be exhaust grids, exhaust holes, or a combination of exhaust holes with the noise-reducing pieces 7. When the exhaust channel 4 are exhaust grids (as shown in FIG. 9B), the exhaust grids are covered around the side wall of the exhaust connector 5. The exhaust channel can be made from materials such as polyvinyl chloride, polypropylene, polytetrafluoroethylene, or nylon. The connection between the exhaust channel and the first connector 51 can be directly and permanently secured by adhesives, ultrasonic pressing, heat pressing, etc., enhancing the tightness of the connection between the exhaust grids and the exhaust connector 5. When the exhaust channel 4 are exhaust holes (as shown in FIG. 9A), droplet-shaped holes are evenly distributed at regular intervals on the exhaust connector 5. The orientation of the droplet-shaped exhaust holes aligns with the extension direction of the tubular body of the first end 22, with the larger caliber of the holes positioned closer to the fixed opening 11. The configuration of positioning the larger caliber closer to the fixed opening 11 shortens the distance that the gas travels from the fixed opening 11 to the exhaust channel 4, thereby improving the efficiency of waste gas discharge. The distribution and size of the exhaust holes are not fixed. In other embodiments, exhaust holes of different shapes, such as square or triangular, can be employed. The design of the exhaust holes enhances the convenience of establishing the exhaust channel 4 without adding the additional assembly, effortlessly facilitating waste gas expulsion while reducing the weight of the frame assembly and promoting timely gas exchange for the user. When the exhaust channel 4 are the combination of the exhaust holes and the noise-reducing pieces 7, the noise-reducing pieces 7 are simply added at the location of the exhaust holes. The noise-reducing pieces 7 cover the exhaust holes and encases the side wall of the exhaust connector 5, thereby diminishing the sound of the user's exhaled gas flow. According to tests conducted by methods in ISO 17510:2015, the sound decibel level is at or between 40 to 50 decibels when using the exhaust holes alone. After adding the noise-reducing pieces 7, the sound decibel level drops to at or between 19 to 30 decibels, achieving a noise reduction at or between 10 to 30 decibels. The frame assembly assumes a good noise-reducing effect. The noise-reducing pieces 7 can be noise-reducing cotton or noise reducing mesh. The noise-reducing cotton can be made from one of the materials: polyester, polypropylene, polyethylene, nylon, vinylon, or natural fabrics. The noise-reducing mesh can be made from one or more of the materials: polyvinyl chloride, polypropylene, polytetrafluoroethylene, or nylon.

Implementing the frame assembly directly connected to a frame and a short tube of this disclosure has several beneficial effects, including but not limited to:

1. Reduce parts and weight. Most mask systems in the market connect the patient interface pad to other tubular connectors through a combination of the frame, elbow, conduit, and straps, forming a complete gas delivery channel, and delivers pressurized gas to the patient's airway, achieving the purpose of preventing sleep apnea. When wearing the mask system for extended periods at night, the weight of the system may press down on the user, leading to discomfort for some users and causing impressions on the skin, psychological stress, and other sleep disturbances. Therefore, our product innovatively constructs the frame assembly using only three parts—the frame, the gas delivery hose, and side straps. Combined with the redesigned nasal pillow, a completely new nasal mask system is developed. In the new frame assembly, the frame is designed to connect directly with the gas delivery hose. The connection of the exhaust channel on the elbow and the frame is redesigned, eliminating the traditional elbow part and configuring an exhaust channel on the frame which can be combined with noise-reducing materials. This not only reduces the noise of the product, ensuring a quiet environment for users during use but also lightens the overall weight of the assembly, making it more comfortable to wear, thereby enhancing the comfort and overall experience of the user.

2. Simplify user operation. The connection between the gas delivery hose and the frame is configured to be detachable, streamlining the installation process. This allows users to quickly disassemble and wear the mask system, preventing scenarios where users need to move quickly, such as waking up at night or in emergencies, but can't promptly separate the mask from the machine. Also, the second end of the gas delivery hose can rotate 360°. Compared to the current permanent connection method between the gas delivery hose and the frame, the detachable and rotatable design reduces the likelihood of the hose getting tangled due to being fixed at both ends. This greatly enhances the flexibility and adaptability of the frame assembly. Furthermore, the detachable hose offers users a more convenient option for cleaning and replacement. Users can carry out regular maintenance and cleaning, ensuring the mask system remains in good condition and avoiding potential health risks.

3. More economical and environmentally friendly for users. The application of the detachable gas delivery hose brings numerous economic and environmental advantages. Given the vulnerability of the hose material compared to other parts of the frame assembly, this detachable design allows users to replace only the damaged part, such as the gas delivery hose or other parts, without replacing the entire frame assembly. This reduces operational and maintenance costs and prolongs the overall system's lifespan. It enhances the durability and reusability of the gas delivery hose, making maintenance and repair of the entire frame assembly more efficient, cost-effective, and environmentally friendly.

4. Ease of Manufacturing for Producers. From the production perspective, the use of the detachable connection structure for the assembly makes the manufacturing process more convenient, avoiding the common issues of damage in traditional welding or fixed connections, which is often encountered in the production of integrated frame assemblies and gas delivery hoses. As a result, the defect rate is significantly reduced. When maintenance is required or damaged components need to be replaced, only simple mechanical disassembly is needed. This process allows for quick inspection and repair, ensuring the continuity and stability of the production process. Through such straightforward mechanical structures, the efficiency of the production line is maintained, providing a more efficient and reliable production process for manufacturers.

5. Enhanced comfort in use. To improve comfort when wearing, special attention is given to the design of the side connectors. To adapt to facial deformities, a deformable material with inherent support properties selected and an integrated injection molding process is used to prevent any joint marks that can cause discomfort on the face. The overall side connector has a smooth arc, fitting more closely to the user's facial deformities, offering a more comfortable wearing experience. Multiple connection options are provided, including buckles, snap fasteners, hook and loop fasteners, rotary knobs, and magnetic attraction fasteners, for flexible connection with the frame. To further enhance the product's usability, “L/R” embossed signs, indicating left and right, are marked on the side connectors, assisting users to quickly and easily wear the mask even in dark environments, providing a more intelligent and convenient user experience.

In summary, our product innovatively constructs the frame assembly as a combination of three parts: the frame, the gas delivery hose, and the side connectors. While retaining the product's primary functions, it achieves an enhanced user experience, improving the overall comfort of the product, making it easier to clean, and significantly extending its overall lifespan. Moreover, the detachable design effectively reduces production costs and actively contributes to environmental protection. The disassembling structure combination and manufacturing process make the production process more efficient and economical, minimizing resource waste. Additionally, its detachable and reusable features contribute to reducing waste, making it more environmentally friendly. Overall, the design of this frame assembly directly connected to a frame and a short tube not only enhances functionality and user experience but also balances comfort and environmental considerations, offering users a more convenient, efficient, and sustainable user experience.

The above descriptions of the disclosure's embodiments are illustrated with reference to the accompanying drawings, but the disclosure is not limited to these specific embodiments. The described embodiments are merely illustrative and not restrictive. It must be noted that as used herein and in the appended claims, the regular forms “a” “an” “the” include their plural equivalents, unless the context clearly dictates otherwise. Those skilled in the art can make various modifications and variations under the guidance of this disclosure, without departing from the scope and spirit protected by the claims of this disclosure. All such modifications and variations are within the scope of the disclosure's protection.

1. A frame assembly comprising:
a frame for securing a nasal pillow,
a gas delivery hose directly connected to the frame for delivering breathable gas to the nasal pillow, and
side connectors for securing the frame to a user's face;
wherein
the frame is configured to accommodate the nasal pillow and position the nasal pillow at a user's nostrils with at least a part of the frame being rigid, and the frame is configured to bend towards the user's face such that the frame fits to the user's face and ensures a secure attachment of the nasal pillow to the user's face;
the frame including:
a fixed opening located in a middle of the frame, wherein the frame connects to the gas delivery hose through the fixed opening, and the fixed opening is a tubular body of a certain length, providing a contact surface for connection of the gas delivery hose to the fixed opening;
first connection parts located horizontally on both sides of the frame with the frame connecting to the side connectors via the first connection parts;
the gas delivery hose including:
a first end located at an upper part of the gas delivery hose for detachable connection to the frame, wherein the gas delivery hose is configured to rotate relative to the frame once the gas delivery hose is connected to the frame;
a middle hose positioned between the first end and a second end, and connected to both the first end and the second end, the middle hose having multiple continuous evenly folded walls, enabling the middle hose to bend and stretch;
the second end located at a lower part of the gas delivery hose and configured to connect with an other tubular connector; the second end consisting of a first part and a second part, wherein the first part is fixedly connected to a lower end of the middle hose, with an other end of the first part away from the middle hose connected to the second part, and an other end of the second part connects with the other tubular connector, thereby being configured to deliver breathable gas to the gas delivery hose, which is then channeled through the frame and the nasal pillow to the user's nostrils; wherein
the side connectors connect to the first connection parts on both sides of the frame; and the side connectors consists of a front end and a rear end, the front end configured to connect with the frame, while the rear end is configured to make contact with the user's face;
the frame further including an exhaust channel, wherein
the exhaust channel is located on the frame and configured to allow for expulsion of exhaled gas from the user's nostrils through the nasal pillow, and the exhaled gas then flows into the frame and exits via the exhaust channel on the frame, and
wherein a protrusion and a corresponding recess for receiving the protrusion are provided on an inner wall of the first end and an outer wall of the fixed opening, and
wherein the fixed opening has a central axis located in a middle of the fixed opening, the frame is a symmetrical shape with left-right symmetry along a straight line which is an axis of symmetry for the frame, and the central axis and the axis of symmetry have at least one of the following characteristics: A. an angle α, formed by the central axis of the fixed opening and the straight line connecting a furthest point on the frame from the fixed opening and a center point defined by the central axis of the fixed opening, is at or between 0° to 90° towards the user's face; B. an angle β, formed by the axis of symmetry of the frame and the central axis of the fixed opening, is at or between 30° and 150°.

2. (canceled)

3. The frame assembly according to claim 1, wherein a length of the gas delivery hose is at or between 200 to 600 mm, an external diameter of the gas delivery hose is between 25 to 45 mm, and a wall thickness of the gas delivery hoses is at or between 1 to 5 mm, with an expandability of the gas delivery hose being at or between 80% to 120%.

4. The frame assembly according to claim 1, wherein at least one of the connections between the gas delivery hose and the frame, the front end of the side connectors and the frame, and the nasal pillow and the frame are connected by at least one connector that includes one or more of buckles, snap fasteners, hook and loop fasteners, rotary knobs, or magnetic attraction fasteners.

5. The frame assembly according to claim 1, wherein there are multiple exhaust channels on the frame, and a combined area of the exhaust channels constitutes 20%-50% of an outer surface area of the frame.

6. The frame assembly according to claim 1, wherein the exhaust channel is positioned towards a side of the gas delivery hose, includes a noise reduction piece that covers the exhaust channel and is configured to reduce noise when the frame vents to an external environment through the exhaust channel.

7. The frame assembly according to claim 1, wherein the exhaust channel includes exhaust grids or exhaust holes, and the exhaust grids have through holes opened on a surface of the frame and are attached to the frame to cover the through holes by adhesive, buttons, ultrasonic bonding, or heat pressing; and the exhaust holes are formed by multiple small circular holes on the surface of the frame.

8. The frame assembly according to claim 1, wherein the second part of the second end of the gas delivery hose is a cylinder that is configured to rotate coaxially relative to the first part, allowing the second part to achieve coaxial rotation relative to the gas delivery hose.

9. The frame assembly according to claim 1, wherein the front end and rear end of the side connectors are integrally formed through injection molding, and a linear distance between a center point of the front end and a center point of the rear end is at or between 20 to 200 mm.

10. The frame assembly according to claim 6, wherein the noise reduction piece includes one or more of noise-reducing cotton or noise-reducing mesh, wherein the noise-reducing cotton is made from at least one material selected from polyester, polypropylene, polyethylene, nylon, vinylon, and natural fabric; and wherein the noise-reducing mesh is made from at least one material selected from polyvinyl chloride, polypropylene, polytetrafluoroethylene, or nylon.

11. A frame assembly comprising:
a frame for securing a nasal pillow,
a gas delivery hose directly connected to the frame for delivering breathable gas to the nasal pillow, and
side connectors for securing the frame to a user's face, wherein
the frame is configured to accommodate the nasal pillow and position the nasal pillow at a user's nostrils with at least a part of the frame being rigid, and the frame is configured to bend towards the user's face such that the frame fits to the user's face and ensures a secure attachment;
the frame including:
a fixed opening located in a middle of the frame, wherein the frame connects to the gas delivery hose through the fixed opening, and the fixed opening is a tubular body of a certain length, providing a contact surface for connection of the gas delivery hose to the fixed opening;
first connection parts located on two sides of the frame in a same horizontal direction, wherein the frame connects to the side connectors through the first connection parts, and the gas delivery hose is configured to rotate relative to the frame when the gas delivery hose is connected to the frame;
the gas delivery hose including:
a first end located at an upper part of the gas delivery hose, wherein the first end is configured for detachable connection to the frame and the first end has an exhaust channel to allow airflow from the gas delivery hose to an external environment;
a middle hose positioned between the first end and a second end to connect with the first end and the second end, wherein the middle hose has multiple continuous evenly folded walls that allow for bending and stretching of the middle hose;
the second end located at a lower part of the gas delivery hose and configured to connect with an other tubular connector; the second end consisting of a first part and a second part,
wherein the first part is fixedly connected to a lower end of the middle hose, with an other end of the first part away from the middle hose connected to the second part, and an other end of the second part connects with the other tubular connector, thereby being configured to deliver breathable gas to the gas delivery hose, which is then channeled through the frame and the nasal pillow to the user's nostrils; wherein
the side connectors are provided in a pair, connecting to the first connection parts on both sides of the frame; and the side connectors have a front end and a rear end, the front end connecting to the frame, and the rear end is configured to make contact with the user's face;
the frame further including: an exhaust channel; wherein
the exhaust channel is located on the first end of the gas delivery hose, and configured to allow gas exhaled from a user's nose to be directed to the nasal pillow when the nasal pillow is used, the gas flowing from the nasal pillow through the frame into the gas delivery hose, and the exhaled gas is expelled through the exhaust channel on the first end of the gas delivery hose,
wherein a protrusion and a corresponding recess for receiving the protrusion are provided on an inner wall of the first end and an outer wall of the fixed opening, and
wherein the fixed opening has a central axis located in a middle of the fixed opening, the frame is a symmetrical shape with left-right symmetry along a straight line which is an axis of symmetry for the frame, and the central axis and the axis of symmetry have at least one of the following characteristics: A. an angle α, formed by the central axis of the fixed opening and the straight line connecting a furthest point on the frame from the fixed opening and a center point defined by the central axis of the fixed opening, is at or between 0° to 90° towards the user's face; B. an angle β, formed by the axis of symmetry of the frame and the central axis of the fixed opening, is at or between 30° and 150°.

12. (canceled)

13. The frame assembly according to claim 11, wherein a length of the gas delivery hose is at or between 200 to 600 mm, an external diameter of the gas delivery hose is between 25 to 45 mm, and a wall thickness of the gas delivery hoses is at or between 1 to 5 mm, with an expandability of the gas delivery hose being at or between 80% to 120%.

14. The frame assembly according to claim 11, wherein one or more connections between the gas delivery hose and the frame, the side connectors and the frame, and the nasal pillow and the frame are connected by at least one connector that includes one or more of buckles, snap fasteners, hook and loop fasteners, rotary knobs, or magnetic attraction fasteners.

15. The frame assembly according to claim 11, wherein there are multiple exhaust channels on the first end of the gas delivery hose, and a combined area of the exhaust channels constitutes 20%-50% of an outer surface area of the first end of the gas delivery hose.

16. The frame assembly according to claim 11, wherein a side of the exhaust channel facing towards the external environment, includes a noise reduction piece that covers the exhaust channel and is configured to reduce noise when the gas delivery hose vents to the external environment through the exhaust channel.

17. (canceled)

18. The frame assembly according to claim 11, wherein the second part of the second end of the gas delivery hose is a cylinder that is configured to rotate coaxially relative to the first part, allowing the second part to achieve coaxial rotation relative to the gas delivery hose.

19. (canceled)

20. The frame assembly according to claim 16, wherein the noise reduction piece includes one or more of noise-reducing cotton or noise-reducing mesh, wherein the noise-reducing cotton is made from at least one material selected from polyester, polypropylene, polyethylene, nylon, vinylon, and natural fabric; and wherein the noise-reducing mesh is made from at least one material selected from polyvinyl chloride, polypropylene, polytetrafluoroethylene, or nylon.

21. A frame assembly comprising:
a frame for securing a nasal pillow,
a gas delivery hose connected to the frame for delivering breathable gas to the nasal pillow, and
side connectors for securing the frame to a user's face; wherein
the frame is configured to accommodate the nasal pillow and position the nasal pillow at a user's nostrils with at least a part of the frame being rigid, and the frame is configured to bend towards the user's face such that the frame fits to the user's face and ensures a secure attachment;
the frame including:
a fixed opening located in a middle of the frame, wherein the frame connects to the gas delivery hose through the fixed opening, and the fixed opening is a tubular body of a certain length, providing a contact surface for connection of the gas delivery hose to the fixed opening;
first connection parts located on two sides of the frame in a same horizontal direction, wherein the frame connects to the side connectors through the first connection parts;
the gas delivery hose including:
a first end located at an upper part of the gas delivery hose for detachable connection to the frame, wherein the gas delivery hose is configured to rotate relative to the frame once the gas delivery hose is connected to the frame;
a middle hose positioned between the first end and a second end to connect with the first end and the second end, wherein the middle hose has multiple continuous evenly folded walls that allow for bending and stretching of the middle hose;
the second end located at a lower part of the gas delivery hose and configured to connect with an other tubular connector; the second end consisting of a first part and a second part, wherein the first part is fixedly connected to a lower end of the middle hose, with an other end of the first part away from the middle hose connected to the second part, and an other end of the second part connects with the other tubular connector, thereby being configured to deliver breathable gas to the gas delivery hose, which is then channeled through the frame and the nasal pillow to the user's nostrils; wherein
the side connectors are connected to the first connection parts on both sides of the frame; and the side connectors have a front end and a rear end, the front end connecting to the frame, and the rear end is configured to make contact with the user's face;
the frame further including: an exhaust channel and an exhaust connector; wherein
the exhaust channel is located on the exhaust connector, and is configured to allow a user to exhale gas from the user's nostrils to the nasal pillow, then the exhaled gas flows sequentially through the nasal pillow to the frame and the exhaust connector, and then vented out through the exhaust channel on the exhaust connector;
the exhaust connector including a first connector and a second connector, wherein the first connector is configured to connect with the fixed opening of the frame, and the second connector is configured to connect with the first end of the gas delivery hose,
wherein a protrusion and a corresponding recess for receiving the protrusion are provided on an inner wall of the first end and an outer wall of the fixed opening, and
wherein the fixed opening has a central axis located in a middle of the fixed opening, the frame is a symmetrical shape with left-right symmetry along a straight line which is an axis of symmetry for the frame, and the central axis and the axis of symmetry have at least one of the following characteristics: A. an angle α, formed by the central axis of the fixed opening and the straight line connecting a furthest point on the frame from the fixed opening and a center point defined by the central axis of the fixed opening, is at or between 0° to 90° towards the user's face; B. an angle β, formed by the axis of symmetry of the frame and the central axis of the fixed opening, is at or between 30° and 150°.

22. (canceled)

23. The frame assembly according to claim 21, wherein a length of the gas delivery hose is at or between 200 to 600 mm, an external diameter of the gas delivery hose is between 25 to 45 mm, and a wall thickness of the gas delivery hoses is at or between 1 to 5 mm, with an expandability of the gas delivery hose being at or between 80% to 120%.

24. The frame assembly according to claim 21, wherein one or more connections between the gas delivery hose and the frame, the side connectors and the frame, and the nasal pillow and the frame are connected by at least one connector that includes one or more of buckles, snap fasteners, hook and loop fasteners, rotary knobs, or magnetic attraction fasteners.

25. The frame assembly according to claim 21, wherein there are multiple exhaust channels on the exhaust connector, and a combined area of the exhaust channels constitutes 20%-50% of an outer surface area of the exhaust connector.

26. The frame assembly according to claim 21, wherein the exhaust channel has a noise reduction piece which covers an exhaust position of the exhaust channel to reduce noise when the exhaust connector vents to an external environment through the exhaust channel.

27. The frame assembly according to claim 26, wherein the exhaust channel includes exhaust grids or exhaust holes, wherein the exhaust grids are made of through holes opened on a surface of the exhaust connector and are attached to the exhaust connector to cover the through holes by adhesive, buttons, ultrasonic bonding, or heat pressing; and the exhaust holes are formed by multiple small circular holes on the surface of the exhaust connector.

28. (canceled)

29. The frame assembly according to claim 21, wherein the front end and rear end of the side connectors are integrally formed through injection molding, and a linear distance between a center point of the front end and a center point of the rear end is at or between 20 to 200 mm.

30. (canceled)

31. A frame assembly comprising:
a frame for securing a nasal pillow,
a gas delivery hose directly connected to the frame for delivering breathable gas to the nasal pillow, and
side connectors for securing the frame to a user's face;
wherein
the frame is configured to accommodate the nasal pillow and position the nasal pillow at a user's nostrils with at least a part of the frame being rigid, and the frame is configured to bend towards the user's face such that the frame fits to the user's face and ensures a secure attachment of the nasal pillow to the user's face;
the frame including:
a fixed opening located in a middle of the frame, wherein the frame connects to the gas delivery hose through the fixed opening, and the fixed opening is a tubular body of a certain length, providing a contact surface for connection of the gas delivery hose to the fixed opening;
first connection parts located horizontally on both sides of the frame with the frame connecting to the side connectors via the first connection parts;
the gas delivery hose including:
a first end located at an upper part of the gas delivery hose for detachable connection to the frame, wherein the gas delivery hose is configured to rotate relative to the frame once the gas delivery hose is connected to the frame;
a middle hose positioned between the first end and a second end, and connected to both the first end and the second end, the middle hose having multiple continuous evenly folded walls, enabling the middle hose to bend and stretch;
the second end located at a lower part of the gas delivery hose and configured to connect with an other tubular connector; the second end consisting of a first part and a second part, wherein the first part is fixedly connected to a lower end of the middle hose, with an other end of the first part away from the middle hose connected to the second part, and an other end of the second part connects with the other tubular connector, thereby being configured to deliver breathable gas to the gas delivery hose, which is then channeled through the frame and the nasal pillow to the user's nostrils; wherein
the side connectors connect to the first connection parts on both sides of the frame; and the side connectors consists of a front end and a rear end, the front end configured to connect with the frame, while the rear end is configured to make contact with the user's face;
the frame further including an exhaust channel, wherein
the exhaust channel is located on the frame and configured to allow for expulsion of exhaled gas from the user's nostrils through the nasal pillow, and the exhaled gas then flows into the frame and exits via the exhaust channel on the frame;
wherein an angle is formed between an axis of symmetry of the frame and a central axis of the fixed opening,
wherein a protrusion and a corresponding recess for receiving the protrusion are provided on an inner wall of the first end and an outer wall of the fixed opening, and
wherein the fixed opening has a central axis located in a middle of the fixed opening, the frame is a symmetrical shape with left-right symmetry along a straight line which is an axis of symmetry for the frame, and the central axis and the axis of symmetry have at least one of the following characteristics: A. an angle α, formed by the central axis of the fixed opening and the straight line connecting a furthest point on the frame from the fixed opening and a center point defined by the central axis of the fixed opening, is at or between 0° to 90° towards the user's face; B. an angle β, formed by the axis of symmetry of the frame and the central axis of the fixed opening, is at or between 30° and 150°.

32. The frame assembly according to claim 31, wherein the certain length of the fixed opening is between 0.3 mm to 30 mm.

33. The frame assembly according to claim 31, wherein the angle between the axis of symmetry of the frame and the central axis of the fixed opening is between 30° to 150°.

34. The frame assembly according to claim 31, wherein the frame further includes an exhaust connector.
